(19) United States
(12) Patent Application Publication
(10) Pub. No.: US 2001/0034373 A1
(43) Pub. Date: Oct. 25, 2001

(54) Low dose modafinil for enhancement of cognitive function

(76) Inventors: Matthew Miller, Newtown, PA (US); Patricia C. Contreras, San Diego, CA (US)

Correspondence Address: CEPHALON, INC., 145 BRANDYWINE PARKWAY, WEST CHESTER, PA, 19380-4245, US

(21) Appl. No.: 09/779,417

(22) Filed: Feb. 8, 2001

Related U.S. Application Data
(60) Provisional application No. 60/181,283, filed on Feb. 9, 2000.

(51) Int. Cl. A61K 31/16

(52) U.S. Cl. 514/625

(57) ABSTRACT

Modafinil is shown to be effective in improving or restoring cognitive function in the humans or other mammals when administered at doses that are substantially lower than optimal wakefulness-promoting doses. Daily dosages of less than 100 mg/day and more particularly from about 1 to about 75 mg/day are shown to be effective.

### BACKGROUND OF THE INVENTION

1. Field of the Invention

The present invention is related to the field of neuropharmacological agents, including agents that affect the cognitive functions, and more particularly to the improvement of cognitive function in a subject in which cognitive function has been diminished.

2. Description of Related Art

Modafinil (C₁₅H₁₅NO₂S), 2-(benzhydrylsulfinyl) acetamide, or 2-[(diphenylmethyl)sulfinyl] acetamide, is a synthetic acetamide derivative with wake-promoting activity, the structure of which has been described in French Patent No. 78 05 510 and in U.S. Pat. No. 4,177,290, and which has been approved by the United States Food and Drug Administration for use in the treatment of narcolepsy. Modafinil was tested in combination with various agents including apomorphine, amphetamine, reserpine, oxotremorine, hypnotics, yohimbine, 5-hydroxytryptophan, monoamine oxidase inhibitor (I.M.A.O.), and in several behavioral conditions, as described in the cited patents. A method of preparation of a racemic mixture is described in the '290 patent and a method of preparation of a levorotatory isomer is described in U.S. Pat. No. 4,927,855 (both incorporated herein by reference). The levorotatory isomer is reported to be useful for treatment of hypersonmia, depression, Alzheimer's disease and to have activity towards the symptoms of dementia and loss of memory, especially in the elderly. However, these patents do not appear to describe any effect of the levorotatory isomer on a loss of cognitive function due to Alzheimer's Disease, nor do they appear to describe any effects of a low dose modafinil as described herein below.

Modafinil has also been described as an agent with activity in the central nervous system, and as a useful agent in the treatment of Parkinson's disease (U.S. Pat. No. 5,180,745); in the protection of cerebral tissue from ischemia (U.S. Pat. No. 5,391,576); in the treatment of urinary and fecal incontinence (U.S. Pat. No. 5,401,776); and in the treatment of sleep apneas and disorders of central origin (U.S. Pat. No. 5,612,378). U.S. Pat. No. 5,618,845 describes modafinil preparations of a defined particle size less than about 200 microns that is more potent and safer than preparations containing a substantial proportion of larger particles.

Various pharmacological agents have been described for treatment of cognitive dysfunction. U.S. Pat. No. 5,602,131 describes ebumane analogs, heterocyclic inducers of tyrosine hydroxylase (TH). TH is an enzyme that controls the synthesis of the transmitter in the central catecholaminergic and dopaminergic neurons, and because the speed of synthesis of this transmitter is associated with the appearance of tonic dysfunctions of the brain, these compounds are proposed as therapeutic agents in the treatment of disorders such as anxiety, psychoses, depression, memory disorders in the course of senescence and/or degenerative diseases and in Parkinson's disease. Other pharmacological agents that have been suggested for treatment of cognitive dysfunction include heterocyclic compounds such as 3,4-diphenyl chromans (U.S. Pat. No. 5,756,539), tacrine metabolites (U.S. Pat. No. 5,767,126), and aza-cyclic compounds (U.S. Pat. Nos. 5,773,452; 5,919,805; and RE 35,822), all of which are incorporated herein by reference. The compounds described in these patents appear to have in common the ability to directly or indirectly affect cholinergic neurotransmitters, or the postsynaptic muscarinic receptors for cholinergic neurons. Other proposed therapies for loss of cognitive function include polyamine compounds for protection of overstimulation of NMDA receptors (U.S. Pat. No. 5,886,051) and a lipid soluble preparation of thiamine to improve glucose metabolism in the brain (U.S. Pat. No. 5,885,608).

There is still a need, however, for an effective treatment for the restoration of cognitive function that is non-toxic and effective for long-term administration in order to improve the quality of life, and productivity of humans and animals subject to cognitive dysfunction due to advanced age or disease.

### SUMMARY OF THE INVENTION

The present disclosure provides novel compositions of modafinil and methods of using these compositions that are effective at improving cognitive function in a subject in need thereof It has been surprisingly found that administration of modafinil at doses much lower than the approved doses known to promote wakefulness improves cognitive function in aged rats to levels comparable to young controls. It is a further surprising discovery that this effect is not seen at doses of 200 to 400 mg/day, normally used to promote wakefulness, or to treat excessive daytime sleepiness (EDS) associated with narcolepsy, for example. As used herein, the term “improving cognitive function” would encompass activating, increasing, stabilizing, maintaining, enhancing or restoring cognitive function in a subject.

The present invention may be described in certain embodiments as a composition comprising a modafinil compound for administration to a mammal, said composition including from about 1 mg to about 75 mg of the modafinil compound. A modafinil compound as described herein may include a racemic compound, and may be in an acid form, such as a metabolic acid of modafinil or a benzhydrylsulfinylacetic acid, a sulfone form, a hydroxylated form, a conjugated form such as a modafinil compound conjugated to a protein, a polysaccharide, a glucuronide or a sulfate, or a polymorphic form. In certain embodiments the composition will include from about 1 mg to about 75 mg, from about 5 to about 60 mg, from about 25 to about 40 mg, or even about 30 to 35 mg of the modafinil compound, wherein in certain preferred embodiments the modafinil compound is modafinil.

It is also preferred that the composition as described above contain a single daily dose for administration to a mammal, and including a mammal that has a loss of cognitive function. Preferred compositions will take various forms, but the most preferred are oral pharmaceutical preparations, and more particularly tablets for oral administration. Such a tablet will contain the active ingredient, a modafinil compound or modafinil, for example, and may also include any or all of the following inert ingredients: lactose, corn starch, magnesium silicate, croscarmellose sodium, povidone, magnesium stearate, or talc.

The compositions, preparations and pharmaceutical preparations described herein may be formulated and/or adapted for administration to a variety of preferably mammalian subjects, including veterinary subjects as well as human subjects. Certain preparations and formulations as described herein will be beneficial in the treatment of loss of cognition in human Alzheimer's disease patients, Age-Related Cognitive Decline patients, or animal or human patients suffering or susceptible to a temporary or permanent loss of cognitive function, for example, due to advanced age, trauma, stress, disease, schizophrenia, or transient impairment due to chemical imbalance or toxicity, such as ethanol toxicity.

As such, the present invention also provides a method of preparing a composition for improvement of cognitive function in a subject in need thereof, where the method includes combining from about 1 to about 75 mg, about 5 to about 60 mg, about 15 to about 50 mg, about 25 to about 40 mg, or even about 30 to 35 mg of modafinil with a pharmaceutically acceptable carrier. The composition may be adapted as described above, and preferably is prepared as an oral pharmaceutical preparation, or more particularly a tablet for oral administration as described above.

Another preferred embodiment of the invention is a method of improving a cognitive function in an animal or human subject, where the method includes administering to the subject an amount of modafinil effective to improve the cognitive function. As shown herein, an effective amount of modafinil may be substantially lower than the optimum dose effective to promote wakefulness in a subject, and is preferably lower than 200 mg/day in an adult human. It is a surprising aspect of the present invention that the optimal wakefulness promoting doses are much less effective at improving cognitive function than are lower doses. An aspect of the invention is, therefore, a method for treating impaired cognition in a mammal susceptible to the development of or suffering from cognition loss, the method including administering to a mammal amounts of a modafinil compound effective to improve cognition in the animal, where the amounts of modafinil compound being administered periodically in unit doses are substantially lower than optimum wakefulness promoting dosages in the mammal. As described elsewhere herein, the optimum wakefulness promoting dose for an adult human is about 200 mg/day. As such, preferred unit doses provide daily doses of from about 1 mg to about 75 mg orally, or the oral dose necessary to achieve a serum level of modafinil of from about 0.05 to about 2 μg/ml.

It is also an aspect of the present invention that a modafinil compound such as modafinil may be combined with other pharmaceutical agents, and more particularly, with agents that are useful for the treatment of impaired cognition associated with various disease states including, for example, age, trauma, stress or transient impairment due to chemical imbalance or toxicity, hypersomnia, depression, Alzheimer's Disease, non-Alzheimer's dementias, including Lewy body dementia, vascular dementia and binswanger's dementia, and schizophrenia. The present invention would encompass, therefore, combinations of modafinil or a modafinil compound with ebumane analogs, heterocyclic inducers of tyrosine hydroxylase, 3,4-diphenyl chromans, tacrine metabolites, aza-cyclic compounds, polyamine compounds, or thiamine; nonanticholinergic antidepressants such as benzodiazepines; phenothiazines aliphatic such as chlorpromazine; piperidines such as thioridazine; piperazines such as trifluoperazine, fluphenazine and perphenazine; dibenzoxazepines such as loxapine; dihydroindolones such as molindone; thioxanthenes such as thiothixene; butyrophenones such as haloperidol; diphenylbutyl-piperidines such as pimozide; dibenzodiazepine such as clozapine; benzisoxazole such as risperidone; thienobenzodiazepine such as olanzapine; dibenzothiazepine such as quetiapine; imidazolidinone such as sertindole and benzisothiazolyl-piperazine such as ziprasidone.

It is an object of the present disclosure also, to provide in a method for using a modafinil compound in mammals as a medicine, an improvement which includes administering modafinil in unit doses that are (a) substantially lower than the optimal unit doses that are effective to promote wakefulness in the mammal and (b) effective to stabilize or improve cognition in the mammal, and preferably where the mammal is susceptible to the development of or is suffering from cognition loss.

The present disclosure also provides a pharmaceutical composition in unit dose form, for use in treating loss of cognition in a mammal susceptible to the development of or suffering from cognition loss, which includes an amount of a modafinil compound such that one or more unit doses thereof are effective to stabilize or improve cognition in the mammal upon periodic administration and the unit doses being administered periodically are substantially lower than an optimum dosage effective to promote wakefulness in the mammal. Such a pharmaceutical composition would preferably include the modafinil compound contained in each unit dose that provides a stable serum level of about 0.05 to about 2 μg/ml or more preferably, from about 0.1 to about 1.5 μg/ml of the modafinil compound upon daily administration of one or more unit doses, and may include a tablet for oral administration.

The present disclosure also provides a therapeutic package for dispensing to, or for use in dispensing to, a mammal being treated for loss of cognition, the package including (1) one or more unit doses, each such unit dose containing an amount of a modafinil compound such that said one or more unit doses thereof are effective to stabilize or improve cognition in said animal upon periodic administration and the unit doses being administered periodically are substantially lower than a minimal optimum dosage effective to promote wakefulness in said mammal, and (2) a finished pharmaceutical container therefor, said container containing (a) said unit dose or unit doses and (b) labeling directing the use of said package in the treatment of said mammal. In preferred embodiments the unit dose is adapted for oral administration.

The present disclosure also includes, in certain embodiments, a method of treating dementia, or even dementia due to Alzheimer's disease in a human subject in need thereof, where the method includes administering to the subject a composition including modafinil in a daily dose that is effective to substantially improve the dementia and is lower than the minimal optimum daily dose that is effective to promote wakefulness in the human subject. Such a dose would, in certain embodiments include an oral dosage of less than 200 mg and more preferably from about 5 to about 75 mg.

The present discovery may be described in certain aspects, therefore, as a composition comprising a low dose of modafinil for use in improving cognitive function in a subject in need thereof, such as a subject that has an age-related loss of cognitive function. In certain embodiments, an effective dose is from about 5 to about 75 mg, from about 10 to about 60 mg, from about 15 to about 50 mg, or about 30 to 35 mg. A composition as described herein may be an oral pharmaceutical preparation, or even a tablet for oral administration.

Also described herein are methods of preparing a composition for improvement of cognitive function in a subject in need thereof including combining an effective low level dose of modafinil as described in the preceding paragraph with a pharmaceutically acceptable carrier. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions comprising modafinil as described herein may be orally administered with an inert diluent or an assimilable edible carrier, for example. The compositions may also be enclosed in hard or soft shell gelatin capsule, compressed into tablets, or incorporated directly with the food of the diet. For oral therapeutic administration, modafinil may be incorporated with excipients and used in the form of ingestible tablets, buccal tablets, troches, capsules, elixirs, solutions, suspensions, syrups, wafers, dermal patches and the like, although sustained release formulations are the generally preferred method of administering modafinil. Such compositions and preparations should contain at least 0.1% of active compound. The percentage of the compositions and preparations may, of course, be varied and may conveniently be between about 2 to about 60% of the weight of the unit.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring, for example. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained or variable release rate preparations and formulations. Variable release rate formulations are those in which the rate of release of the active agent varies over the dissolution time of a single dose of the formulation.

In certain embodiments, the disclosed compositions may be formulated to be administered by use of a skin patch, or transdermal delivery system. The administration of the modafinil compositions described herein transdermally may be accomplished by any of a number of systems known in the art. Examples of systems that may be adapted for use with the compositions described herein include those systems of transdermal administration described in U.S. Pat. No. 4,816,252; U.S. Pat. No. 5,122,382; U.S. Pat. No. 5,198,223; U.S. Pat. No. 5,023,084; U.S. Pat. No. 4,906,169; U.S. Pat. No. 5,145,682; U.S. Pat. No. 4,624,665; U.S. Pat. No. 4,687,481; U.S. Pat. No. 4,834,978; and U.S. Pat. No. 4,810,499 (all incorporated herein by reference).

These methods typically include an adhesive matrix or drug reservoir system and may include a skin permeation enhancement agent such as ethanol, polyethylene glycol 200 dilaurate, isopropyl myristate, glycerol trioleate, linolenic acid saturated ethanol, glycerol monooleate, glycerol monolaurate, n-decyl alcohol, capric acid, and certain saturated and unsaturated fatty acids, and their esters, alcohols, monoglycerides, acetate, diethanolamides and N,N-dimethylamides (See for examples, U.S. Pat. No. 4,906,169).

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is data from delayed alternation studies in rats, where open squares indicate the percent correct responses in young controls, open diamonds indicate percent correct responses in aged controls, and filled triangles indicate aged rats administered modafinil at 30 mg/kg.

FIG. 2 is data from delayed alternation studies in rats, where open squares indicate the percent correct responses in young controls, open diamonds indicate percent correct responses in aged controls, and x's indicate aged rats administered modafinil at 100 mg/kg.

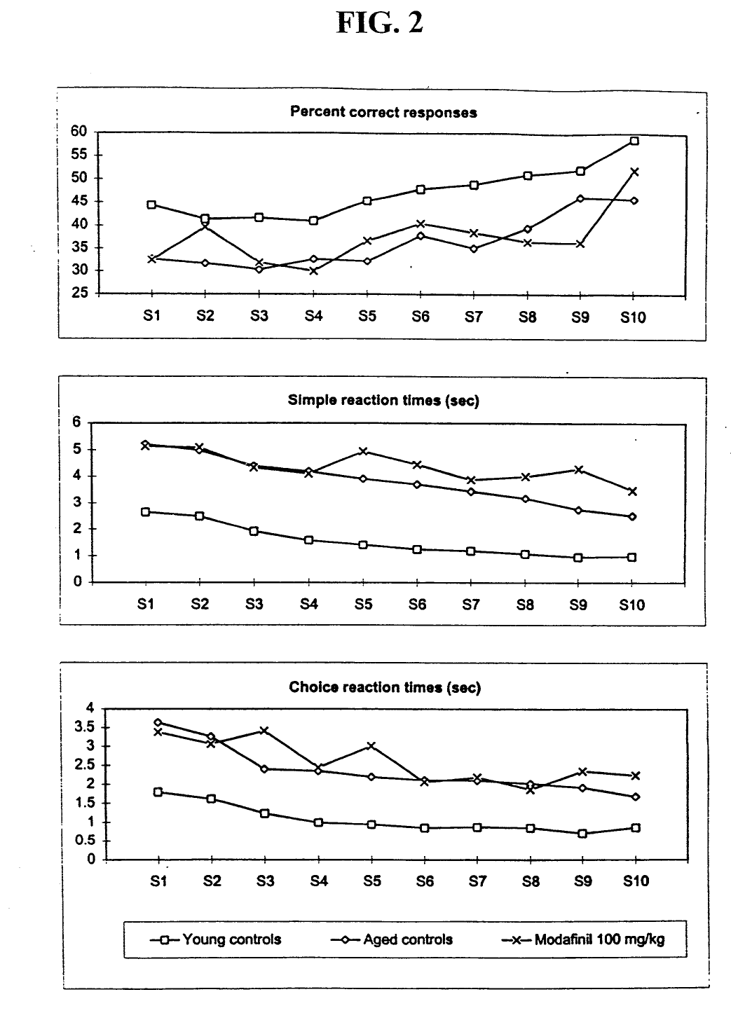

### DESCRIPTION OF PREFERRED EMBODIMENTS

The present invention arises from the discovery that modafinil, when administered at low doses, is able to restore cognitive function in aged rats to levels seen in young rats. As shown in studies described herein, aged rats show clear deficits in cognitive ability when compared to young, matched control rats in a delayed alternation task. This age-related loss of cognitive ability is also shown herein to be distinguished from an impairment in short term memory. Another surprising discovery as disclosed herein is that the enhancement of cognitive function in aged rats occurs at dosages of modafinil that are lower than those known to produce wakefulness, and that at the normal wake-promoting doses, the enhancement in cognitive function disappears. These data suggest that the cognitive activity of modafinil arises from a different mechanism or target site than the wakefulness promoting activity.

Cognition is a general term understood in the art as the quality of knowing, and includes perceiving, recognizing, conceiving, judging, sensing, reasoning, and imagining, (See Stedman's Medical Dictionary, 25^{th} edition, Williams & Wilkins, Baltimore). Disorders of cognition, or cognitive dysfunction may be characterized as forgetfulness, confusion, memory loss, attentional deficits, affective or emotional disturbances, and deficits in learning, association, consolidation and recognition, and would include the condition known as Age-Related Cognitive Decline. As such, an improvement in cognitive function as demonstrated in the studies described herein, is a novel use for modafinil containing compositions. In addition to novel methods of use, modafinil is shown herein to be effective at improving cognitive function at low doses with respect to those shown to be effective in wake-promoting activity. The present discovery, therefore, represents a new and surprising use for modafinil and also provides for compositions that include modafinil at lower dosages than the optimal effective doses that are commonly used for narcolepsy or hypersomnia, for example.

It has been previously shown that aged rats (>18 months) show clear deficits as compared with young rats in the acquisition of an operant delayed alternation task. Delayed alternation tasks have been suggested as a potentially useful tool in the study of age-related memory deficits in rats (Porsolt et al., 1995, Drug Development Research 35:214). This task requires the rat to learn to alternate its responses between two levers in a standard Skinner Box in order to obtain a food reward. After having previously acquired a single lever-pressing response, the animal is given a series of trials consisting of the presentation, via retractable levers, of a left or right lever, followed 5 seconds later by the presentation of the two levers. The animal must learn to press the lever opposite to that which was presented 5 seconds previously (delayed alternation) to obtain food.

The delayed alternation procedure measures drug effects on response accuracy (percent correct responses), attention (simple reaction time in a one lever presentation and response omissions), and information processing (choice reaction time in the two lever presentation). The response accuracy is calculated as the number of reinforced, or correct responses (delayed alternations) expressed as a percent of the total number of responses emitted in a choice response between two levers. This measure is an indication of the learning capacities of the animal during acquisition. The simple reaction time is a measurement of the time elapsed between the presentation of a lever at the beginning of a trial and the moment when the animal pressed it. The time is expressed as the mean for the total number of trials initiated. This value indicates the general attention capacity of the animal to respond to an unpredictable spatial stimulus. The choice reaction time is the time elapsed between the presentation of the two levers and the moment when the animal pressed a lever. This data is presented as the mean for the total number of trials completed. This measure indicates the information processing speed of the animal. Response failures are the number of times an animal fails to respond to either the single lever or to both levers. Aged animals show deficits on all parameters. Modafinil, administered at doses of 60 mg/kg or less restored cognitive function in the aged rats to the level of the young control rats.

In another study, aged rats were tested for performance of a stabilized delayed alternation task at three retention delays. After stabilized performance in training sessions the rats were entered into a drug testing study with 15, 30 or 60 mg/ml of modafinil. A trial started with the presentation of a single lever (left or right). A response on this lever resulted in the retraction of the lever from the chamber, the delivery of a food pellet and started a retention delay (5, 10 or 20 seconds). After the expiration of the delay, the 2 levers were inserted into the chamber and the animal received a food pellet only if it pressed the lever not previously presented (delayed alternation). Incorrect responses (responses on the same lever) were not reinforced. The 3 delays were randomly presented and equally distributed throughout the session. Correct or incorrect responses were followed by retraction of the two levers. If, during a one-lever or two-lever presentation, the animal did not press a lever within 20 seconds, the lever(s) were withdrawn without food reward (response omission). The test results indicated the response accuracy of the animal after each retention delay, the simple reaction time in the one lever presentation, and the choice reaction time in the two lever presentation. In these studies, aged rats showed a clear decrease in response accuracy as a function of the retention delay, and modafinil did not significantly affect the number of correct responses at any retention delay. Therefore, modafinil had no effect on improving short term memory at these doses.

An aspect of the present invention is the surprising activity of modafinil at lower dosages than has been observed to have an effect on wakefulness or alertness. The dosage levels in rats in the studies described herein are reported in mg/kg administered by intraperitoneal injection (i.p.). As described in previous publications, the wakefulness promoting dose of modafinil in rats is highly significant at doses of 100 to 300 mg/kg i.p. (Edgar and Seidel, The Journal of Pharmacology and Experimental Therapeutics, vol. 283, pp 757-769, 1997). The extrapolation in adult humans is a wakefulness promoting dose of 200 mg/day. A dose of 200 mg to an adult human would result in a serum level of about 6.4 μg/ml. Therefore, the doses as described herein to improve cognitive function would be most effective when the concentration of modafinil in the blood is between about 0.05 and about 2 μg/ml. An effective dose, therefore, is a dose given as a single unit per day in a sustained release formula or given periodically would be that dose that maintains a serum level of from about 0.05 to about 2 μg/ml.

Prior to any invention disclosed or claimed herein, modafinil was known in the art in the form of a therapeutic package, marketed under the name Provigil®. Provigil® is a pharmaceutical product marketed by Cephalon, Inc. of West Chester, Pa. Provigil® is supplied as tablets containing 100 mg or 200 mg modafinil. In commercial use, modafinil-containing therapeutic packages in the prior art were labeled and otherwise indicated for use in narcolepsy patients.

Accordingly, known in the prior art were therapeutic packages providing one or more unit doses of modafinil as an active ingredient thereof, supplied in a finished pharmaceutical container that contain said unit doses, and further contained or comprised labeling directing the use of said package in the treatment of a human disease or condition as described above. In the provided literature accompanying a pharmaceutical container are instructions that the daily dosage of modafinil is 200 mg/day given as a single dose in the morning.

In particular, the package insert approved by the FDA for the product Provigil®, marketed by Cephalon, Inc. indicates the availability of 100 and 200 mg modafinil tablets. For example, the package insert indicates the availability of 100 and 200 mg. modafinil tablets. The package insert further describes the manner in which modafinil-containing therapeutic packages were supplied for commercial use or sale. The package insert cited above provides an example of the complete approved labeling t0hat comprised a part of the known therapeutic packages indicated for narcolepsy. The complete material may also be found, at the time of filing of the present application, on the World Wide Web at http://www.provigil.com.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

Modafinil was evaluated after i.p. administration in aged male Wistar rats, 23 months old, to evaluate its effects on the acquisition of an operant delayed alternation task for food reward. After 3 weeks of lever-pressing training (single lever), each animal was submitted to a daily delayed alternation acquisition session (two levers) in a Skinner box for 2 consecutive weeks (10 sessions). In the delayed alternation acquisition sessions the animals were given 35 trials, each consisting of the presentation of one lever followed 5 seconds later by the presentation of the two levers. The animals had to respond on the lever not previously presented (delayed presentation). Five measures were taken: percent correct responses (response accuracy), simple and choice reaction times and the number of failures to respond to one or two levers (omissions). Modafinil (30, 100 and 300 mg/kg) was administered i.p. 30 minutes before each delayed alternation acquisition session, and compared with a 23 month-old aged control group and a 2 month-old young control group. Eleven to 12 animals per group were used at the beginning of the drug testing period.

The results of these studies showed that the aged controls, in comparison with young controls, showed deficits in the acquisition and performance of the delayed alternation task. This was indicated by poor response accuracy (impaired learning), slower reaction times and more response omissions (impaired attention). Modafinil (30 mg/kg i.p.) significantly increased the number of correct responses (both weeks) and significantly decreased choice reaction times (second test week). No clear effects were observed on simple reaction times, although a tendency towards a decrease was apparent during the second test week. Modafinil (from 100 to 300 mg/kg i.p.) failed to improve the general ability of animals to perform the task. Indeed, some rats did not complete the minimal criteria of 20 trials during some sessions, therefore decreasing the number of animals retained for data analysis. Modafinil also tended to increase simple reaction times during the second test week and significantly increased the number of response omissions, mainly during the one-lever presentations. No effects were observed at this dose on the number of correct responses.

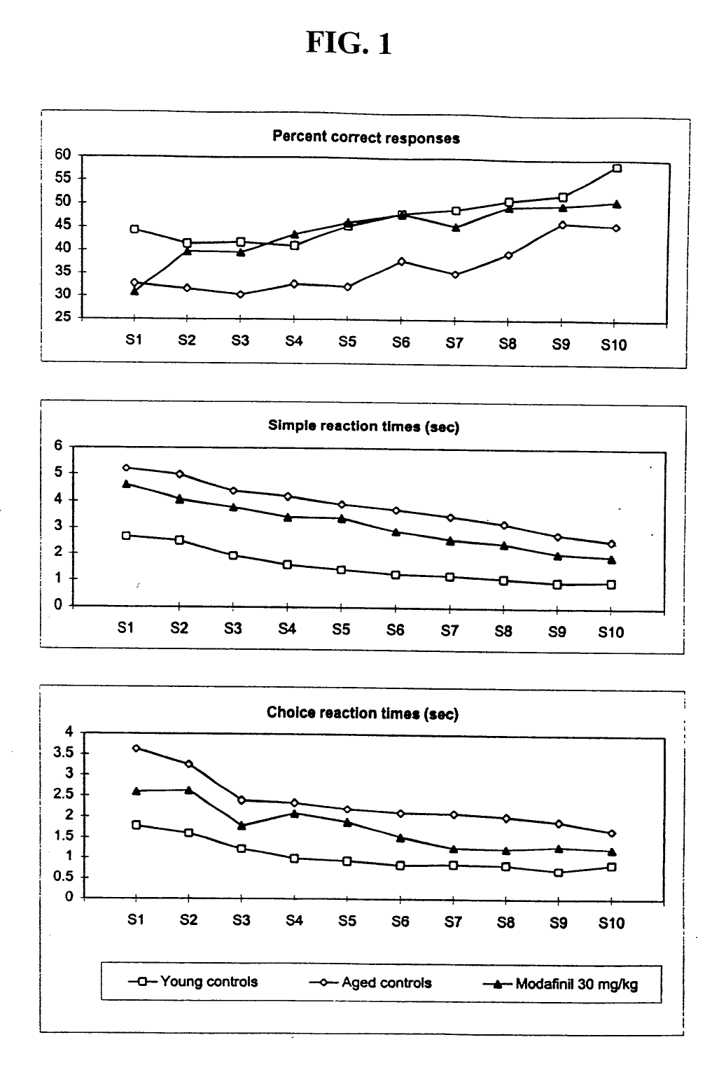

As shown in FIG. 1A, aged rats receiving 30 mg/kg performed as well as the young controls and significantly better than the aged controls in percent correct responses in the delayed alternation test, at least in sessions 2-9. As shown in FIG. 1B, however, this effect does not occur at 100 mg/kg modafinil, a level representing the lower threshold of wake-promoting doses.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A composition comprising a modafinil compound for administration to a mammal, said composition comprising an amount of a modafinil compound effective to improve cognitive function in said mammal.

2. The composition of claim 1, further defined as comprising from about 1 mg to about 75 mg of said modafinil compound.

3. The composition of claim 1, wherein said composition comprises from about 5 to about 60 mg of said modafinil compound.

4. The composition of claim 1, wherein said composition comprises from about 15 to about 50 mg of said modafinil compound.

5. The composition of claim 1, wherein said composition comprises from about 25 to about 40 mg of said modafinil compound.

6. The composition of claim 1, wherein said composition comprises from about 30 to about 35 mg of said modafinil compound.

7. The method according to claim 1, wherein said composition comprises an amount of modafinil compound effective to achieve a serum level in said mammal of from about 0.05 to about 2 μg/ml modafinil when administered to said mammal.

8. The composition of claim 1, wherein said modafinil compound is modafinil.

9. The composition of claim 1, wherein said mammal has or is susceptible to a loss of cognitive function due to age, trauma, stress, disease, or transient impairment due to chemical imbalance or toxicity.

10. The composition of claim 1, wherein said composition comprises an oral pharmaceutical preparation.

11. The composition of claim 1, wherein said composition comprises a tablet for oral administration.

12. The composition of 1, wherein said tablet comprises lactose, corn starch, magnesium silicate, croscarmellose sodium, povidone, magnesium stearate, or talc.

13. The composition of claim 1, wherein said mammal is a human.

14. The composition of claim 1, wherein said mammal is a human Alzheimer's disease, Age-Related Cognitive Decline, or schizophrenia patient.

15. A method of preparing a composition for enhancement of cognitive function in a subject in need thereof comprising combining an amount of a modafinil compound effective to improve cognitive function in said mammal with a pharmaceutically acceptable carrier.

16. The method of claim 15, further defined as comprising combining from about 1 to about 75 mg of modafinil with a pharmaceutically acceptable carrier.

17. The method of claim 15, further defined as comprising combining from about 5 to about 60 mg of modafinil with a pharmaceutically acceptable carrier.

18. The method of claim 15, further defined as comprising combining from about 15 to about 50 mg of modafinil with a pharmaceutically acceptable carrier.

19. The method of claim 15, further defined as comprising combining from about 25 to about 40 mg of modafinil with a pharmaceutically acceptable carrier.

20. The method of claim 15, further defined as comprising combining from about 30 to about 35 mg of modafinil with a pharmaceutically acceptable carrier.

21. The method of claim 15, wherein said subject has or is susceptible to a loss of cognitive function due to age, trauma, stress, disease, or transient impairment due to chemical imbalance or toxicity.

22. The method of claim 15, wherein said composition comprises an oral pharmaceutical preparation.

23. The method of claim 15, wherein said composition comprises a tablet for oral administration.

24. The method of claim 23, wherein said composition comprises lactose, corn starch, magnesium silicate, croscarmellose sodium, povidone, magnesium stearate, or talc in any combination thereof.

25. A method of improving a cognitive function in an animal or human subject comprising administering to said animal an amount of modafinil effective to improve said cognitive function.

26. The method of claim 25, wherein said subject is a human.

27. The method of claim 25, wherein said effective amount is substantially lower than the equivalent of 200 mg/day in a human adult.

28. A method for treating loss of cognition in a mammal susceptible to the development of or suffering from cognition loss, which comprises administering to said mammal amounts of a modafinil compound effective to stabilize or improve cognition in said mammal.

29. The method of claim 28, wherein said amounts of modafinil compound being administered periodically in unit doses that are substantially lower 200 mg/day dosages in said mammal.

30. The method according to claim 28, wherein said amounts of modafinil compound are effective to achieve a serum level of from about 0.05 to about 2 μg/ml modafinil in said subject.

31. A method according to claim 28 wherein said loss of cognition is associated with age, trauma, stress, disease, or transient impairment due to chemical imbalance or toxicity.

32. A method according to claim 28 wherein said loss of cognition is associated with Age-Related Cognitive Decline, Alzheimer's disease, or schizophrenia.

33. A method according to claim 28 wherein said unit doses provide daily doses of from about 1 mg to about 75 mg orally.

34. A method according to claim 28 wherein said modafinil compound is modafinil.

35. A method according to claim 28 wherein said mammal is a human subject.

36. In a method for using a modafinil compound in mammals as a medicine, the improvement which comprises:
administering said modafinil compound in doses that are (a) substantially lower than the minimal optimal unit doses that are effective to promote wakefulness in said mammal and (b) effective to stabilize or improve cognition in said mammal.

37. An improvement according to claim 36 wherein said mammal is susceptible to the development of or suffering from a loss of cognitive function associated with age, trauma, stress, disease, or transient impairment due to chemical imbalance or toxicity.

38. An improvement according to claim 36 wherein said unit doses provide daily doses effective to achieve a serum level in said mammal of from about 0.05 to about 2 μg/ml.

39. An improvement according to claim 36 wherein said unit doses are administered orally.

40. An improvement according to claim 36 wherein said modafinil compound is modafinil.

41. An improvement according to claim 40 wherein said mammal is a human subject.

42. A pharmaceutical composition in unit dose form, for use in treating loss of cognitive function in a mammal susceptible to the development of or suffering from a loss of cognitive function, which comprises:
an amount of a modafinil compound such that one or more unit doses thereof are effective to stabilize or improve cognitive function in said mammal upon periodic administration.

43. The composition of claim 42, wherein the unit doses being administered periodically are substantially lower than 200 mg.

44. A pharmaceutical composition according to claim 42 wherein the amount of the modafinil compound contained in each unit dose provides a dose effective to achieve a serum level of modafinil in said mammal of from about 0.05 to about 2 μg/ml.

45. A pharmaceutical composition according to claim 42 wherein the amount of the modafinil compound contained in each unit dose provides a dose effective to achieve a serum level of modafinil in said mammal of from about 0.05 to about 1.5 μg/ml.

46. A pharmaceutical composition according to claim 42 wherein the amount of the modafinil compound contained in each unit dose provides a dose effective to achieve a serum level of modafinil in said mammal of from about 0.1 to about 1.5 μg/ml.

47. A pharmaceutical composition according to claim 42 wherein the amount of the modafinil compound contained in each unit dose provides a dose effective to achieve a serum level of modafinil in said mammal of from about 0.5 to about 1.5 μg/ml.

48. A pharmaceutical composition according to claim 42 wherein the amount of the modafinil compound contained in each unit dose provides a dose effective to achieve a serum level of modafinil in said mammal of from about 0.5 to about 1 μg/ml.

49. The pharmaceutical composition according to claim 42 wherein the composition comprises a tablet for oral administration.

50. The pharmaceutical composition according to claim 42, wherein said modafinil compound is modafinil.

51. The pharmaceutical composition according to claim 42, wherein said mammal has or is susceptible to a loss of cognitive function due to age, trauma, stress, disease, or transient impairment due to chemical imbalance or toxicity.

52. The pharmaceutical composition according to claim 48, wherein said tablet comprises lactose, corn starch, magnesium silicate, croscarmellose sodium, povidone, magnesium stearate, or talc.

53. The pharmaceutical composition according to claim 42, wherein said mammal is a human.

54. The pharmaceutical composition according to claim 42, wherein said mammal is a human Alzheimer's disease, Age-Related Cognitive Decline, or schizophrenia patient.

55. A therapeutic package for dispensing to, or for use in dispensing to, a mammal being treated for loss of cognition, comprising:
(1) one or more unit doses, each such unit dose containing an amount of a modafinil compound such that said one or more unit doses thereof are effective to stabilize or improve cognition in said mammal upon periodic administration and the unit doses being administered periodically are substantially lower than 200 mg/day in said mammal, and
(2) a finished pharmaceutical container therefor, said container containing (a) said unit dose or unit doses and (b) labeling directing the use of said package in the treatment of said mammal.

56. A therapeutic package according to claim 55 wherein the unit dose is adapted for oral administration.

57. A method of treating dementia in a human subject in need thereof, said method comprising administering to said subject a composition comprising modafinil in a daily dose that is effective to substantially improve said dementia and is lower than the minimal optimum daily dose that is effective to promote wakefulness in said human subject.

58. The method according to claim 56, wherein said daily dose is an oral dosage of from about 1 to about 75 mg.

59. A method of treating dementia due to Alzheimer's disease in a human in need thereof, said method comprising administering to said subject a composition comprising modafinil in a daily dose that is effective to substantially improve said dementia and is lower than 200 mg/day.

60. The method according to claim 46, wherein said daily dose is an oral daily dosage of from about 1 to about 75 mg.
